(19) United States
(12) Patent Application Publication
(10) Pub. No.: US 2025/0194756 A1
(43) Pub. Date: Jun. 19, 2025

(54) MULTISENSOR BRACELET

(71) Applicant: Legionarius, LLC, Wilmington, DE (US)

(72) Inventor: Alexander GRUENTZIG, Sudbury, MA (US)

(21) Appl. No.: 18/980,973

(22) Filed: Dec. 13, 2024

Related U.S. Application Data
(60) Provisional application No. 63/610,201, filed on Dec. 14, 2023.

(51) Int. Cl. A44C 5/00; A61B 5/00

(52) U.S. Cl. CPC A44C 5/0015; A61B 5/0015; A61B 5/681; A61B 5/742; A61B 5/746; A61B 5/7475

(57) ABSTRACT

Featured are bracelets including a geolocation sensor and one or more physiological sensors. The physiological sensors may be selected from a heart rate sensor, an oxygen saturation sensor, a blood pressure sensor, a respiratory rate sensor, a temperature sensor, a vital sign monitoring (VSM) sensor, an impact detection sensor, a position sensor, a contact sensor, a hydration sensor, an electrolyte sensor, and a movement sensor. The geolocation sensor and/or the one or more physiological sensors may be integrated within the bracelet. In some embodiments, the geolocation sensor and/or the one or more physiological sensors may be configured to be activated upon contact with a user and/or deformation of the bracelet.

### BACKGROUND OF THE INVENTION

Hemorrhage from vascular injuries in the proximal extremities, pelvis, and abdomen is extremely difficult to triage in the field outside of medical facilities. While the treatment of such injuries is challenging when they occur in civilian populations, they are even more difficult to treat in combat or mass casualty situations for humans and nonhuman animals alike. While improvements in body armor have reduced mortality from combat injuries to the chest, the incidence of penetrating injuries to the extremities and their associated mortality remain high. Devices have been developed to protect a person from and/or treat injuries sustained in combat situations. However, when the person or animal is injured, the appropriate parties are not always promptly notified of the injury or the nature of the injury. The time between injury and proper care is a critical window that can significantly affect the outcome. If the window is too long, the person or animal may die. Accordingly, new technologies are needed that can be used in order to relay critical information to a person or nonhuman animal wearing the device or to a third party responder(s), such as a medical professional.

### SUMMARY OF THE INVENTION

In one aspect, the invention features a bracelet including a geolocation sensor and one or more physiological sensors. The physiological sensors may be selected from a heart rate sensor, an oxygen saturation sensor, a blood pressure sensor, a respiratory rate sensor, a temperature sensor, a vital sign monitoring (VSM) sensor, an impact detection sensor, a position sensor, a contact sensor, a hydration sensor, an electrolyte sensor, and a movement sensor. The geolocation sensor and/or the one or more physiological sensors may be integrated within the bracelet, e.g., as opposed to being housed in a separate module. The geolocation sensor and/or the one or more physiological sensors may be internal sensors. The geolocation sensor and/or the one or more physiological sensors may be external sensors, e.g., embedded on an external portion of the bracelet. In some embodiments, geolocation sensor and/or the one or more physiological sensors may be configured to be activated upon contact with a user and/or deformation of the bracelet.

In another aspect, the invention features a bracelet including a geolocation sensor and one or more physiological sensors. The physiological sensors may be selected from a heart rate sensor, an oxygen saturation sensor, a blood pressure sensor, a respiratory rate sensor, a temperature sensor, a vital sign monitoring (VSM) sensor, an impact detection sensor, a position sensor, a contact sensor, a hydration sensor, an electrolyte sensor, and a movement sensor. The geolocation sensor and/or the one or more physiological sensors may be configured to be activated upon contact with a user and/or deformation of the bracelet.

In another aspect, the invention features a bracelet that is configured as a collar, e.g., for a non-human subject, such as a dog. The collar may include a geolocation sensor and/or one or more physiological sensors. The physiological sensors may be selected from a heart rate sensor, an oxygen saturation sensor, a blood pressure sensor, a respiratory rate sensor, a temperature sensor, a vital sign monitoring (VSM) sensor, an impact detection sensor, a position sensor, a contact sensor, a hydration sensor, an electrolyte sensor, and a movement sensor. The geolocation sensor and/or the one or more physiological sensors may be integrated within the collar, e.g., as opposed to being housed in a separate module. The geolocation sensor and/or the one or more physiological sensors may be internal sensors. The geolocation sensor and/or the one or more physiological sensors may be external sensors, e.g., embedded on an external portion of the collar. In some embodiments, geolocation sensor and/or the one or more physiological sensors may be configured to be activated upon contact with a user and/or deformation of the collar.

In some embodiments, the bracelet is configured as a wrap. For example, in some embodiments, the bracelet includes an extended configuration and a coiled configuration, e.g., having a first end and a second end opposite the first end. The first end of the bracelet may be configured to partially overlap with the second end of the bracelet upon coiling around an appendage (e.g., a neck, leg, or arm) of a user. The bracelet may include a layer that includes a flexible material (e.g., a deformable or shape memory materials, e.g., a metal, e.g., deformable or shape memory metal) shaped with a width-wise arc. The layer may be disposed in a linear orientation in the extended configuration or a coiled configuration. The layer is disposed in a coiled orientation in the coiled configuration. The bracelet is configured to adopt the coiled orientation by deforming the width-wise arc. A bottom surface may be defined by a convex side formed by the width-wise arc, and a top surface may be defined by a concave side formed by the width-wise arc. The bracelet may further include an outer layer that covers the flexible layer. The outer layer may be composed of or include, for example, fabric, leather, silicone, or plastic. In some embodiments, one of the ends of the bracelet (e.g., the first end) includes a grip tab.

The bracelet may be configured to be worn on any appendage of a subject, such as a neck, arm, or a leg of the subject. For example, the bracelet may be configured to be worn on a wrist or ankle of the subject. The bracelet may lack a clasp or other mechanism that fixedly fastens the bracelet to a user, thus allowing the bracelet to be easily attached to or removed from an appendage.

In some embodiments, the bracelet is configured without straps. For example, the bracelet may be configured with a clip or hook and loop fastener (e.g., VELCRO®) instead of straps. In some embodiments, the bracelet is configured to be mounted on (e.g., clipped to or attached to) a pocket or a wearable device as described herein, e.g., of a human or nonhuman animal, e.g., mounted on a body armor vest.

The bracelet described herein includes a geolocation sensor. The geolocation sensor may be or include a global positioning satellite (GPS) module. The bracelet may further include a button that activates the geolocation sensor and/or the one or more physiological sensors. The button may be disposed on a top surface of the bracelet (e.g., on an end of the wrap, e.g., on a top surface of the first end of the wrap).

In some embodiments, the bracelet further includes a transmitter that transmits data from the geolocation sensor and the one or more physiological sensors. The transmitter may be, for example, a radio frequency transmitter, a cellular transmitter, a WiFi transmitter, an adaptive network topology (ANT) transmitter, a satellite transmitter, or a Bluetooth transmitter. The data may be compiled into a digital casualty care profile card, e.g., prior to transmission.

The bracelet may further include or be a radio node. The radio node may be configured to be integrated with a local area network (LAN) topology, such as a mesh network.

The bracelet may further include an orientation sensor. The orientation sensor may be configured to provide an audio or visual stimulus when the bracelet is adorned in a correct orientation on a subject. The visual stimulus may include, for example, a colorimetric light emitting diode (LED). For example, the colorimetric LED may turn from red to orange or green to indicate that the bracelet is adorned in the correct orientation on the subject.

The bracelet may include one or more colorimetric LEDs (e.g., green, black, red, or yellow/orange) that indicate a health state of the subject.

The bracelet may also include an audio or visual recording device, e.g., to record or transmit a message. The bracelet may be configured to interact with an end-user peripheral device, such as a smart phone or a tablet. The end-user peripheral device may be in close proximity to the bracelet and/or operated by the subject wearing the bracelet. Alternatively, the end-user peripheral device may be located at a distance away from the bracelet and/or operated by a third party user, such as a medical responder.

The bracelet may further include a unique identifier. The unique identifier may be a marking (e.g., a number) on the device or may be a digital identifier. For example, when the bracelet is adorned on a subject, the unique identifier may display or be configured to transmit identification information about the subject wearing the bracelet. The identification information may include one or more of name, age, date, time, unit, blood type, and allergy of the subject. The unique identifier may be linked to the subject's department of defense identification number, name, or call sign.

In another aspect, the invention features a bracelet as described herein, e.g., of any of the above embodiments, and an end-user peripheral device. The bracelet is connected (e.g. wired or wirelessly connected) to the end-user peripheral device. The end-user peripheral device may include a display; one or more processors coupled to the display; and a non-transient memory storing instructions that, when executed by the one or more processors, causes the one or more processors to perform one or more operations. The operations may include rendering a graphical user interface in the display; processing sensor data to produce physiological data; receiving an input of physiological data to the graphical user interface; and/or displaying the physiological data on the graphical user interface. The peripheral device may be configured for wired or wireless communication with one or more sensors of the bracelet.

The physiological data may be selected from one or more of heart rate, heart rate variability, oxygen saturation, blood pressure, respiratory rate, temperature, vital signs, injury status, position, body orientation, and movement status. The peripheral device may be configured to display the physiological data to a third-party responder.

In another aspect, the invention features kit that includes the bracelet of any of the above embodiments or a plurality of the bracelets. The kit may further include an end-user peripheral device. In some embodiments, the bracelet is wirelessly connected to the end-user peripheral device. Each of the plurality of the bracelets may be independently wirelessly connected to the end-user peripheral device.

In another aspect, the invention features a system that includes a plurality of the bracelets, each of which is independently wirelessly connected to an end-user peripheral device or a plurality of end-user peripheral devices. For example, the system may be used by a team, where each member of the team has a bracelet. The system may further include one or more radios, e.g., to be used by the team members.

In another aspect, the invention features a method of using a bracelet as described herein. The method may include presenting physiological data regarding the health state of a subject wearing the bracelet. The method includes acquiring the physiological data from the geolocation sensor and the one or more physiological sensors of the bracelet and presenting the physiological data on an end-user peripheral device. The end-user peripheral device may include a graphical user interface that displays the physiological data, such as one or more of heart rate, heart rate variability, oxygen saturation, blood pressure, respiratory rate, temperature, vital signs, injury status, position, body orientation, and movement status. The graphical user interface may display a geographical location of the subject, e.g., on a map.

The method may further include assigning a health state to the subject wearing the bracelet, e.g., based on the physiological data. The method may include assigning one of four health states to the subject. The four health states may include, for example, (i) black or deceased or near deceased; (ii) red or immediate assistance required; (iii) yellow/orange or delayed response required; and (iv) green or walking wounded. The method may include triaging treatment of the subject by a third-party responder based on the health state of the subject. The method may include alerting a third-party responder to treat the subject.

The bracelet may include an activation button. In such embodiments, the method may include pressing the button prior to or after attaching the bracelet to the user.

The bracelet may include an audio or visual recording device. In such embodiments, the method may include recording an audio or visual message into the device, e.g., by the user or by a third-party responder.

In some embodiments, the bracelet (e.g., collar) contains an electronics module that is operably connected to one or more of the sensors, transmitter, receiver, power source, and any other electronic component of the bracelet. The electronics module (e.g., containing a printed circuit board (PCB)) may be positioned on top of the bracelet. The electronics module may allow the bracelet to function as a hub, e.g., within a radio network (e.g., a mesh node) or for communicating to a peripheral device or a wearable device. The wearable device may be configured for us on a human or a nonhuman subject, such as a dog or horse. The bracelet (e.g., collar) may be configured to interact with a plurality of wearable devices, wherein each member of a team (e.g., a human team member or non-human team member) is adorned with a wearable device. The hub may include one or more processors capable of receiving and processing data from the sensors of the bracelet and/or other peripheral sensors and transmitting the processed data via its own transmitter (e.g., an onboard radio module). The bracelet or a wearable device or other off-the-shelf physiological sensors (e.g., vital sign monitors) could be connected (e.g., wired or wirelessly) to the bracelet, which could serve as a hub, and which could distribute the sensor data (e.g., via a radio frequency transmitter, a cellular transmitter, a WiFi transmitter, an adaptive network topology (ANT) transmitter or ANT+ transmitter, a satellite transmitter, or a Bluetooth transmitter).

### BRIEF DESCRIPTION OF THE DRAWINGS

FIG. 1 is a photograph of a bracelet as described herein.

FIG. 2 is a photograph of an electronics module on the top surface of a bracelet as described herein that is adorned on a subject.

FIG. 3 is an exemplary screenshot of an output on an end-user peripheral device that includes physiological information transmitted to the person operating the end-user peripheral device. The information includes, e.g., identification information, mode of injury (MOI), vital signs, treatment status, fluid status, medications, and events.

FIG. 4 is an exemplary screenshot of an output on an end-user peripheral device that includes physiological information transmitted to the person operating the end-user peripheral device. The information includes heart rate (HR), blood pressure (BP), oxygen saturation (SPO2), and electrocardiogram (ECG).

FIG. 5 is an exemplary screenshot of an output on an end-user peripheral device that includes a position of the subject wearing the bracelet on a map. The screenshot includes the positions of six users, one of which requires assistance, while the other five do not require assistance.

FIG. 6 is a schematic drawing illustrating attachment of the bracelet to a wrist of a subject. The bracelet includes four colorimetric LEDs (circular dots) that correspond to a health status of the subject. The colorimetric LEDs may be, for example, black, red, orange, or green.

FIG. 7 is a schematic drawing showing two team members responding to an injured team member. One of the team members slaps the bracelet on the injured team member, which begins emitting sensor signals from the bracelet.

FIG. 8 is a schematic drawing showing three team members responding to two injured team members. Each injured team member is adorned with a bracelet that emits sensor signals from the bracelet.

FIG. 9 is a schematic drawing showing a team member with a dog wearing a multi-sensor collar.

FIG. 10 is a photograph showing a dog wearing a multi-sensor collar.

FIG. 11 is a a photograph and drawing showing the interaction of a peripheral device with a wearable device on a dog and a wearable device adorned on a human subject.

FIG. 12 is a schematic diagram showing a multi-sensor dog collar used in combination with a peripheral device and a radio to triage treatment for a dog.

### DETAILED DESCRIPTION

Featured are multi-sensor enabled bracelets that are configured to be adorned on a subject (e.g., a human or non-human animal). The multi-sensor enabled bracelet includes a geolocation sensor and one or more physiological sensors that can provide real-time monitoring of health, injury, and casualty information in a digital format. This capability provides a new opportunity for third party responders to be able to monitor casualties and injuries, such as trauma, blood loss, and hemorrhage, triage based on real time accurate information, and save lives during mass casualty events, especially where continuity of care is challenged by unreliable or missing data on previously initiated treatments (e.g., passed on verbally between crews). The bracelet is configured to interact (e.g., wirelessly) with an end-user peripheral device, such as a smartphone, computer, or tablet, to relay this information to the correct person. Furthermore, the bracelet can be configured as a wrap (e.g., a slap wrap) to be quickly adorned on a subject without the need for cumbersome clasps or hooks. Such a bracelet can be attached, for example, to a neck, an arm (e.g., wrist) or leg (e.g., ankle) of a subject in order to transmit real time data. The subject may be a human subject or a nonhuman subject (e.g., a nonhuman animal, such as a mammal, e.g., a dog or a horse). For example, the bracelet may be configured to as a collar to wrap around the neck of the animal. Additionally, one or more of the sensors can be integrated within the bracelet, e.g., as opposed to being housed in a separate module. The geolocation sensor and/or the one or more physiological sensors may be internal sensors. The geolocation sensor and/or the one or more physiological sensors may be external sensors, e.g., embedded on an external portion of the bracelet. In other embodiments, one or more of the sensors may be configured to be activated upon contact with a user and/or deformation of the bracelet. Such a configuration allows the bracelet to be activated upon adorning the bracelet on the subject so that data transmission and/or power usage does not occur until activation.

Also featured are systems including an end-user peripheral device configured to transmit to and receive input from sensors on the bracelet and computer implemented methods using the same. The peripheral devices and systems can receive and process data (e.g., physiological data) from sensors on the bracelet. The devices and systems may utilize software (e.g., an application) running on the devices and systems or accessible from the device or system using a wireless interface, e.g., a cloud-based interface, that communicates with the bracelet. Sensor information processed by the application or a device running the application can be used to provide situational awareness for users under a variety of conditions, such as adverse conditions, e.g., during combat or wartime. This information can be presented to the user of the device, other team members operating in concert (e.g., on a mission) with the user, or a third party.

In the event that an episode (e.g., an injury, such as a catastrophic injury, for example, a ballistic impact) occurs to the user wearing the bracelet, the sensors present on the device can instantly detect various indicia related to the details of the episode (e.g., one or more of heart rate, heart rate variability, oxygen saturation, blood pressure, respiratory rate, temperature, vital signs, injury status, position, body orientation, and movement status). In combat situations, having precise physiological information can provide situational awareness regarding the status of an injured team member, including whether that person requires immediate support or if support can be delayed. The sensor information can be collected, e.g., in real time, and subsequently processed (e.g., by an algorithm e.g., using an application, such as a software application) and displayed to the user (e.g., on a peripheral device worn or operated by the user) or provided to a third party. The peripheral device may be running an application that can process the sensor data into a user-friendly format on a separate device that communicates with the sensors. For example, the sensors may be connected to a transmitter (e.g., a radio frequency transmitter, a cellular transmitter, a WiFi transmitter, an adaptive network topology (ANT) transmitter or ANT+ transmitter, a satellite transmitter, or a Bluetooth transmitter) and/or receiver that transmits the raw data from the sensors that is then processed by the application. The data may be compiled into a digital casualty care profile card, e.g., prior to transmission.

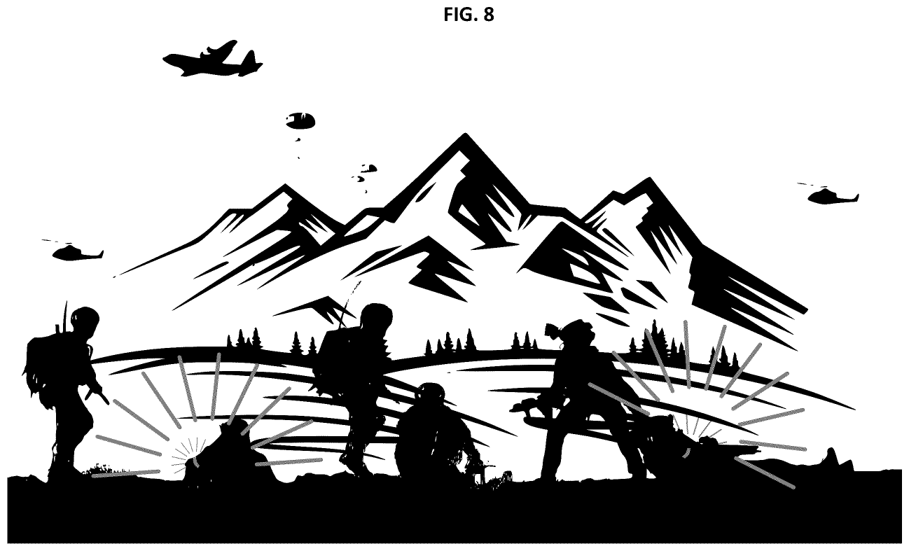

The bracelet may include enhanced communication features. The bracelet may include or be a radio node. The radio node may be configured to be integrated with a local area network (LAN) topology, such as a mesh network. This capability can be enhanced and available in remote areas (e.g., off the grid), e.g., by using a Long Range (LoRa), ANT or ANT+, goTenna Internet of Things (IoT) gateway, or goTenna Pro X mesh gateway, which can significantly extend the range of the bracelet so that it can be backhauled by a command center. The application can then render this information on a GUI (e.g., a touch screen GUI) or transmit the information across the network via one or more radio nodes. This information can be presented to the user and/or distributed to other relevant parties, such as team members or third party responders. The bracelet and peripheral device may be provided as a system or kit that includes plurality of bracelets, e.g., where each bracelet is independently connected (e.g., wired or wirelessly connected) to the end-user peripheral device or a plurality of end-user peripheral devices (FIG. 8). Such a system (optionally including one or more radios) may be used by a team, where each team member has a bracelet. The information may indicate the nature of the impact and/or the health status of the user. This information may be used by a third party to monitor the status (e.g., health status) and, when needed, to provide appropriate response or care to treat the user or triage for later care.

### Bracelet

The multi-sensor enabled bracelet described herein is configured to be quickly adorned on an appendage of a subject, such as a neck, arm, or leg of the subject. For example, the bracelet may be configured to be worn on a wrist or ankle of the subject. The bracelet may lack a clasp or other mechanism that fixedly fastens the bracelet to a user, thus allowing the bracelet to be easily attached to or removed from an appendage without substantial manipulation.

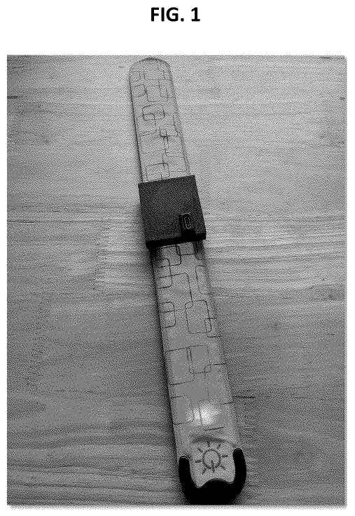

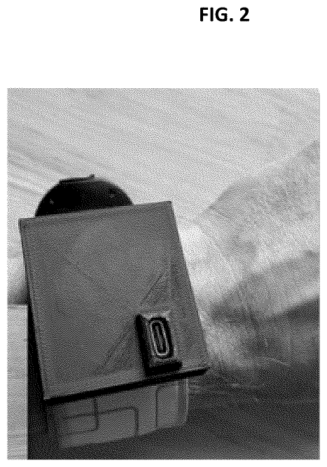

The bracelet may be configured as a wrap, e.g., that coils around the appendage (FIGS. 1 and 2), e.g., a slap wrap. In some embodiments, the bracelet includes an extended configuration and a coiled configuration. For example, the bracelet may include a first end and a second end. The first end of the bracelet may partially overlap with the second end of the bracelet upon coiling around an appendage (e.g., a neck, leg, or arm) of a user. The bracelet may include a layer that includes flexible material (e.g., deformable or shape memory material, e.g., a deformable or shape memory metal) shaped with a width-wise arc. The layer may be disposed in a linear orientation in the extended configuration or a coiled orientation in the coiled configuration. The layer is disposed in a linear orientation in the extended configuration and in a coiled orientation in the coiled configuration by deforming the width-wise arc. A bottom surface may be defined by a convex side formed by the width-wise arc, and a top surface may be defined by a concave side formed by the width-wise arc. Suitable configurations for a wrap bracelet are disclosed, e.g., in US Pub. Nos. US20120324945, US20130044215 and US Design Pat. No. D645,360, the disclosures of which are hereby incorporated by reference in their entirety. The bracelet may be configured to close with a fastener, such as a clasp or buckle. The bracelet may be configured as a collar, e.g., that is worn on a neck of a subject, such as a nonhuman mammal (e.g., a dog or horse).

In some embodiments, the bracelet is configured without straps. For example, the bracelet may be configured with a clip or hook and loop fastener (e.g., VELCRO®) instead of straps. In some embodiments, the bracelet is configured to be mounted on (e.g., clipped to or attached to) a pocket or a wearable device as described herein, e.g., of a human or nonhuman animal, e.g., mounted on a body armor vest.

The bracelet may be suitably sized and shaped to fit on a subject, e.g., on an arm, leg, or neck of a subject. The bracelet may have a square, rectangular, or rounded configuration (e.g., in the extended configuration). The length of the bracelet may be, e.g., from 10 cm to 100 cm, e.g., 10 cm, 20 cm, 30 cm, 40 cm, 50 cm, 60 cm, 70 cm, 80 cm, 90 cm, or 100 cm. The width of the bracelet may be, e.g., from 1 mm to 100 mm, e.g., from 1 mm to 10 mm, e.g., 1 mm, 2 mm, 3 mm, 4 mm, 5 mm, 6 mm, 7 mm, 8 mm, 9 mm, or 10 mm, e.g., from 10 mm to 100 mm, e.g., 10 mm, 20 mm, 30 mm, 40 mm, 50 mm, 60 mm, 70 mm, 80 mm, 90 mm, or 100 mm.

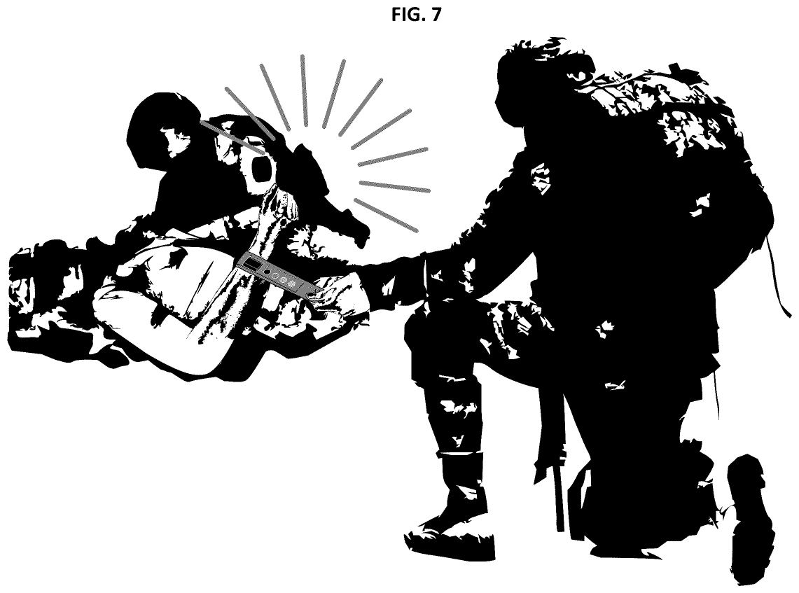

The bracelet may further include an outer layer that covers the flexible layer. The outer layer may be composed of or include, for example, fabric, leather, silicone, or plastic. In some embodiments, one of the ends of the bracelet (e.g., the first end) includes a grip tab. Such a grip tap may allow for facile adornment on a subject, e.g., by slapping the bracelet on an arm or leg of a subject (FIG. 7). The outer layer may include one or more sensors disposed thereon or therein. For example, in one embodiment, one or more of the sensors (e.g., a contact sensor) is integrated within the outer layer or disposed on the surface of the outer layer to sense the user upon contact.

The bracelet may include a unique identifier. The unique identifier may be a marking (e.g., a number) on the device or may be a digital identifier. For example, when the bracelet is adorned on a subject, the unique identifier may display or be configured to transmit identification information about the subject wearing the bracelet. The identification information may include one or more of name, age, date, time, unit, blood type, and allergy of the subject. The bracelet may include an activation button, e.g., to activate the bracelet or the sensors, e.g., prior to or after attaching the bracelet to the user.

The bracelet may also include an audio or visual recording device, e.g., to record or transmit a message. The audio or visual recording device may be used by a third party responder to record a message, e.g., such as a message that is recorded during or following treatment. The audio message may be converted to text that can be displayed on the bracelet (e.g., on a graphical user interface (GUI) or it can be transmitted from the bracelet to a peripheral device). The message may provide information about treatment given to the wearer of the bracelet, treatment that the wearer requires, or information about the health status of the wearer of the bracelet. The information may be used to complete or supplement a casualty care card. The bracelet may be configured to interact with an end-user peripheral device, such as a smart phone or a tablet, e.g., via the audio or visual recording device. The end-user peripheral device may be in close proximity to the bracelet and/or operated by the subject wearing the bracelet. Alternatively, the end-user peripheral device may be located at a distance away from the bracelet and/or operated by a third-party user, such as a medical responder. The bracelet may include a graphical user interface (GUI). The graphical user interface may be used to display an audio or visual message. For example, a text, video, or audio message can be input from a peripheral device and sent to the user wearing the bracelet or a third-party responder to provide treatment instructions.

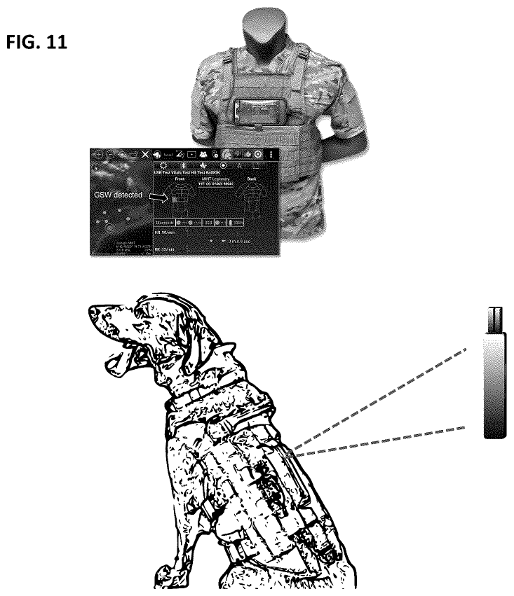

The bracelet may contain an electronics module that is operably connected to one or more of the sensors, transmitter, receiver, power source, and any other electronic component of the bracelet. The electronics module (e.g., containing a printed circuit board (PCB)) may be positioned on top of the bracelet. The electronics module may allow the bracelet to function as a hub, e.g., within a radio network (e.g., a mesh node) or for communicating to a peripheral device or a wearable device (e.g., a wearable device as configured in PCT Pub. Nos. WO 2015/183470 and WO 2018/213615 and US Pub. No. US20200261022, the disclosures of which are hereby incorporated by reference in their entirety (see also FIG. 11). The wearable device may be configured for us on a human or a nonhuman subject, such as a dog or horse. The bracelet (e.g., collar) may be configured to interact with a plurality of wearable devices, wherein each member of a team (e.g., a human team member or non-human team member) is adorned with a wearable device. The collar may be configured to provide an audio, visual, or mechanical or electrical or electronic stimulus to the subject (e.g., to control the animal's behavior from the handler or provide a command).

The bracelet may contain an output device, such as a speaker or an alarm. The output device (e.g., speaker) may be used to communicate with the user or a third party responder, e.g. to send an alarm. For example, a text, video, or audio message can be input from a peripheral device and sent to the user wearing the bracelet or to a third party responder, such as a message providing treatment instructions. The bracelet (e.g., collar) may send an alarm to a handler and/or the handler may send a message to the subject (e.g., dog) to recall the animal (e.g., with a mechanical or electrical stimulus).

The bracelet may be programmed to provide audio or visual guidance to the wearer or a third- party responder regarding medical information (e.g., first aid or triage information), such as in the case of an injury or casualty situation or general health, wellness, and activity tracking information. The information could assist the wearer or third-party responder with assisting another subject or non-human subject or could be information specifically directed to the wearer for their benefit. The bracelet may further include or provide instructions for how to treat a user, e.g., by providing medical guidance on the positioning of the system and/or treatment options. The bracelet may provide guidance (e.g., visual or audio) on first aid steps, how to render aid (e.g., how to use a defibrillator), how to place an item on the subject to be treated, and the like. The bracelet may also allow a third-party responder to connect live to a remote medical professional who can guide the user on the next steps. The bracelet may be used with artificial intelligence to perform triage by itself, e.g., in the case of a mass casualty or injury scenario.

### Sensors

The bracelets described herein include a geolocation sensor and one or more physiological sensors. The geolocation sensor allows the position of the subject wearing the bracelet to be transmitted in real time. The geolocation sensor may be or include a global positioning satellite (GPS) module. The bracelet may further include a button (e.g., disposed on a surface, e.g., top surface of the bracelet) that activates the geolocation sensor and/or the one or more physiological sensors. One or more of the sensors may be integrated within the bracelet. One or more of the sensors may be disposed in a module (e.g., an electronics module) on top of the bracelet (FIG. 2). In some embodiments, the sensors are operably connected to an electronics module (e.g., containing a PCB) that is positioned on top of the bracelet. In some embodiments, one or more of the sensors or other electronics components may be integrated into the bracelet, such that they are fixedly attached and not separatable from the bracelet.

The bracelets described herein include one or more sensors (e.g., biometric or physiological sensors), such as a heart rate sensor, an oxygen saturation sensor, a blood pressure sensor, a respiratory rate sensor, a temperature sensor, a vital sign monitoring (VSM) sensor, an impact detection sensor, a contact sensor, a hydration sensor, an electrolyte sensor, and a movement sensor. The bracelet may further include chemical, biological, radiological, and/or nuclear (CBRNE) sensors. These sensors may transmit information to the peripheral device that is displayed on a GUI on the peripheral device. The sensor data can be processed by the information processing unit and the data can be stored non-transiently and/or transformed into a useful output indicative of the health state (e.g., cognitive function or physiological function) of a subject. The sensors may be powered by a power source or energy unit, and they may send their data to an information processing unit (e.g., in the peripheral device or at a remote location). Physiological sensors may be attached to or located on or within the bracelet and may be operably engaged to the wearer for generating physiological signals corresponding to selected physical conditions of the user. In one embodiment, one or more of the sensors may activate upon contact with the subject or deformation of the bracelet. The data from sensors may be processed by an application, e.g., running on a peripheral device, e.g., to trigger a distress signal. The distress signal may include information corresponding to the physiological signals. For example, the physiological sensor may be a thermometer for measuring the body temperature of the user and the distress signal may include information about the body temperature of the user. The physiological sensor may be a blood pressure meter for measuring the blood pressure of the user and the distress signal may include information about the blood pressure of the user.

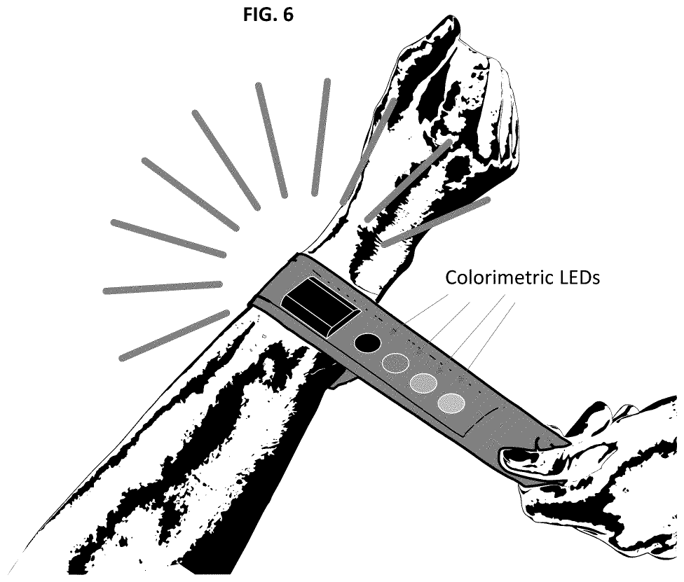

The bracelet may further include an orientation sensor. The orientation sensor may provide an audio or visual stimulus when the bracelet is adorned in a correct orientation on a subject. The visual stimulus may include, for example, a colorimetric light emitting diode (LED). For example, the colorimetric LED may turn from red to orange or green to indicate that the bracelet is adorned in the correct orientation on the subject. One or more of the sensors may not be activated until the contact sensor is activated and/or the bracelet is positioned in the correct orientation. Such a feature can save power by not activating any sensors before the bracelet is being used. In some embodiments, the contact sensor is a temperature sensor that activates upon reaching a predetermined pressure threshold (e.g., body temperature or skin temperature). The bracelet may include one or more colorimetric LEDs that correspond to the health state of the subject. For example, each health state (e.g., (i) black or deceased or near deceased; (ii) red or immediate assistance required; (iii) yellow/orange or delayed response required; and (iv) green or walking wounded) may be denoted by a colorimetric LED or a plurality of colorimetric LEDs on the bracelet, where each LED has a different color (FIG. 6).

The sensors may use electrocardiograma measure heart rate or heart rate variability, a pulse oximeter to measure oxygen saturation levels, or a temperature sensor to measure body temperature. The sensors may be strategically placed near a certain area (e.g., wrist) to track certain physiological parameters associated with a specific area. For example, a sensor or set of sensors can be placed near a vein on the wrist to track heartbeat. The sensors can also be used to transmit information to the user of the device or to a third party upon activation of these sensors (e.g., when a value of the sensor output passes above or below a predetermined threshold). For example, if a set of sensors placed near the wrist detects a drop in heartrate (e.g., with electrocardiograma device can activate to send a distress signal to a third party responder.

The bracelet may be configured with one or more accelerometers, gyroscopes, magnetometers, barometers, relative humidity sensors, bioimpedance sensors, thermometers, biopotential sensors, or optical sensors. Accelerometers (e.g., ADLX345 chip) may be used to track steps, gait, activity, ballistocardiography, heart rate, heart rate volume, relative stroke volume, and respiration rate. A gyroscope (e.g., L3G4200D chip) may be used to track rotation and balance. A magnetometer (e.g., MC5883L chip) may be used to perform magnetoencephalography by recording magnetic currents and electrical circuits. A barometer (e.g., BMP085 chip) may be used to measure pressure. A relative humidity sensor (e.g., Si7023 chip) may be used to measure relative humidity. A bioimpedance sensor (e.g., AFE4300 chip) may be used to measure body composition and EIM. A thermometer (e.g., BMP085 chip) may be used to measure temperature. A biopotential sensor (e.g., HM301D chip) may be used to measure electroencephalography (EEG), electromyography (EMG), echocardiography (EKG), heart rate, heart rate volume, and pulse transit time (blood pressure). An optical sensor (e.g., MAX30100 chip) may be used to measure pulse oxygenation and blood pressure. A photoplethysmography sensor or electrocardiogram (ECG) sensor may be used to track heart rate. A light sensor may be used to measure pulse oximetry (e.g., blood oxygen saturation).

Any of the sensors described above may be configured to transmit various data, e.g., to an information processing unit or a peripheral device. The peripheral device running an application can then use an algorithm to convert the physiological data into biofeedback indicia on a user. The biofeedback indicia may then be rendered on a GUI (e.g., of the peripheral device) for visualization by the user, another user, a central command unit, a team member, or a third party responder. The sensors may track essential vital signs, such as heart rate, blood pressure, orientation, and temperature, to provide critical information for assessing the health state of a user wearing a device containing the sensors. These sensors may be integrated into the device and configured to interact with the peripheral device and/or information processing unit, e.g., by transmitting the biofeedback data (e.g., via Bluetooth, WiFi, Adaptive Network Topology (ANT, e.g., ANT+), Long Range (LoRa, e.g., LoRa MESH), or radio transmission) to the peripheral device, a GUI, or a third party. The device may also be configured to interact in GPS denied environments, e.g., a tunnel. The system may use a sensor such as a camera, star tracker, radar, and/or radio to augment and/or aid inertial navigation system. By communicating these vital positional and biofeedback indicia, the bracelet and/or the peripheral device can provide information, e.g., to a user or a third party responder, about the nature and severity of an impact or injury to a wearer of the device.

### Peripheral Device

A bracelet as described herein can be configured as a system for use with a peripheral device running or accessing software (e.g., an application). The peripheral device may be any suitable medium for computing and/or displaying information. The peripheral device may include any suitable power source to run the device, such as a battery. The peripheral device may be a smartphone (e.g., ANDROID™, IPHONE®), tablet (e.g., IPAD®), computer, cloud-based device (e.g., server), a web-based device, smart glasses, or other information processing device. The peripheral device may be, e.g., a holographic or projected display device (e.g., smart glasses, such as GOOGLE® glass). The peripheral device may be programmed with a software application (e.g., that can be downloaded into the resident memory (e.g., non-transient memory) of the device and run locally on the peripheral device) to receive data that is detected by the sensors on the bracelet and then transmitted (e.g., with a transmitter) to the peripheral device. The peripheral device may include a display with a GUI, one or more processors coupled to the GUI, and a memory (e.g., non-transient memory) storing instructions that, when executed by the one or more processors, causes the one or more processors to perform a programmed operation. This operation may be used to direct an output action (e.g., bladder inflation and signaling for assistance). The operation may include rendering a GUI on a display, receiving an input of data (e.g., ballistic impact data, physiological data, or operational status data) to the GUI, and displaying the data on the GUI. The peripheral device may be configured to receive data from one or more sensors located within or on the bracelet via a wired or wireless connection. Alternatively, or in addition, the device and/or the peripheral device may include a transmitter and/or receiver to transmit the data generated by the sensors to the peripheral device. The transmitter may be, e.g., a smart chip, and can be configured for wired or wireless communication, e.g., through a Bluetooth or Wi-Fi connection, to the peripheral device. The user of the bracelet may use the peripheral device or a third party may use the peripheral device.

The peripheral device may access an application program that provides access to the sensor data via a remote server, e.g., with a cloud-based connection. The bracelet may include a peripheral device, e.g., attached thereto or separate from the device (e.g., as a handheld device). The bracelet may be connected to the peripheral device (e.g., smartphone) running or accessing a software program via a Bluetooth connection. The peripheral device may include a mechanism to read an identification card (e.g., by scanning a barcode or QR code, e.g., on the bracelet) so that important personal information about a user (e.g., medical history, allergies, handicap) is instantly uploaded to the peripheral device running or accessing the application, which can be used to customize the device to a particular user.

Additionally, the peripheral device running the application can be configured to communicate (e.g., through a wired or wireless connection, e.g., through a Bluetooth, Wi-Fi, radio, and/or internet connection) with a database that contains data collected by the bracelet or with another system that receives and processes the data and conveys the information to the peripheral device and/or displays the information on the GUI. Data collected by the bracelet, such as data collected by the sensor(s), may be stored non-transiently in the database, the peripheral device, or other storage medium.

### Application

The peripheral device may be configured to run or access software (e.g., an application). The application may include any suitable computing instructions (e.g., code) that cause the peripheral device to perform an operation. The user of the peripheral device, a third party responder, medical aide, or other relevant personnel may be running the application on his/her peripheral device (e.g., smartphone) to track information about the subject (e.g. human or nonhuman animal) wearing or operating the bracelet (e.g., a collar). For example, the application may be programmed on and/or running locally on the peripheral device. Alternatively, or additionally, the application may not be programmed on and/or running locally on the peripheral device, but rather may be accessed on a remote device (e.g., cloud-based access). The application may include a security feature or login that requires the user to input log-in credentials, e.g., a username or password to access the peripheral device, the application, and/or the cloud-based connection. Exemplary peripheral devices and applications are described, e.g., in US Pub. No. US 20200237318, the disclosure of which is hereby incorporated by reference in its entirety.

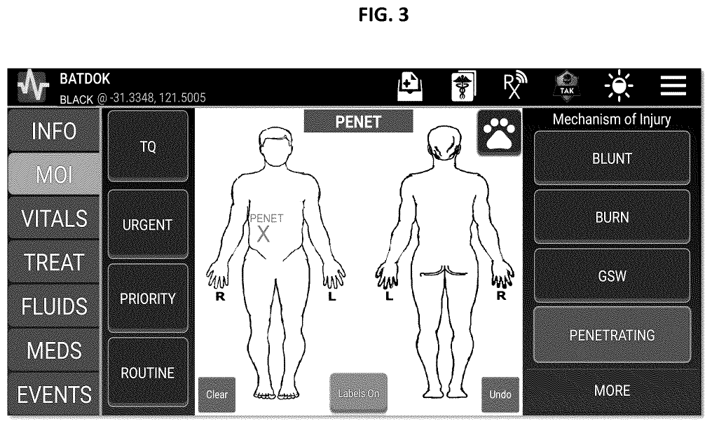

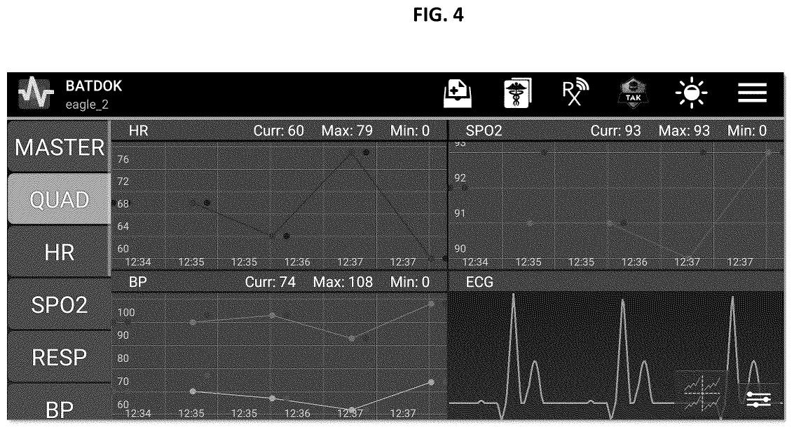

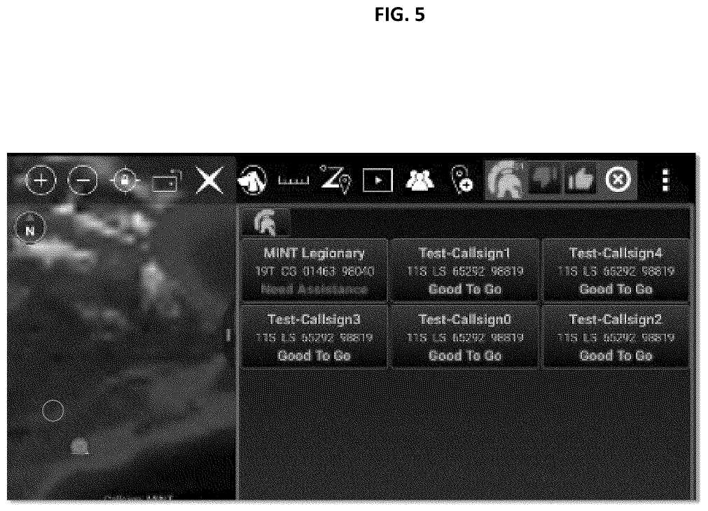

The bracelet may be configured to communicate with a peripheral device, such as a smartphone (e.g., ANDROID™ or IPHONE®) running or accessing an application. The application may be running, e.g., on a holographic or projected display (e.g., smart glasses, such as GOOGLE® glass). The smartphone may be running an ANDROID™ tactical assault kit (ATAK) application, Android Tactical Object Surveillance (ATOS), or a similar application, such as a battlefield assisted trauma distributed observation kit (BATDOK), or Air Force Research Laboratory (AFRL)'s Team Awareness Kit. ATAK is an ANDROID™ smartphone geo-spatial infrastructure application built using NASA World Wind. The application (e.g., ATAK or BATDOK application) provides situational awareness within both civilian and military arenas. The application may have a plugin architecture which allows developers to add or enhance functionality to the application. When used with the bracelet described herein, the application (e.g., ATAK application) can display indicia related to the user or an episode (e.g., catastrophic episode, such as a ballistic impact) experienced by the user, such as projectile impact, acceleration (e.g., moving or still) and orientation (e.g., prone or supine) information of the user, respiration rate, heart rate, user information, and geolocation (see, e.g., FIGS. 3-5). The device may transmit essential physiological indicia and sensor data to the user or to a third party responder using a smartphone running the application (e.g., ATAK application).

The application running on or accessible by the peripheral device may contain features used to control the functionality of the bracelet or the sensors of the bracelet. Some features include a system on/off or reset switch, a power level indicator, the ability to turn certain sensors on or off, or adjust the sensitivity of the sensors. The user of the application can track data from the sensors in real time or observe data over a long time period, and the information may be stored for later analysis. The application may be used to track the health status of an individual, for example, by measuring various parameters, e.g., physiological parameters, such as heart rate or acceleration, or the condition of the individual. The application can be made available for download (e.g., from the internet or the cloud, e.g., from a remote server) on a peripheral device.

The user of the application may adjust the threshold sensitivity of the sensors or whether they trigger an alert upon activation. For example, the sensitivity may be toggled to set a predetermined threshold that is 10% to 100% above a baseline value (e.g., 10%, 20%, 30%, 40%, 50%, 60%, 70%, 80%, 90%, or 100%) before triggering an alert. Examples include adjusting a body temperature sensor to trigger an alert when a body temperature drops by more than 1° C., 2° C., 3° C., 4° C., 5° C., or more. Additionally, details about the nature and location of the stimulus that triggers activation of the device can be displayed on the GUI. Therefore, a first responder would be better prepared upon arrival for treating the injured user. The user of the device can set certain emergency contacts and the emergency contacts can receive a text or SMS message, or a radio signal (e.g., TW-400) upon triggering of the device. The application may further include instructions for how to treat a user, e.g., by providing medical guidance on the positioning of the system and/or treatment options. The application may provide guidance (e.g., visual or audio) on first aid steps, how to render aid (e.g., how to use a defibrillator), how to place an item on the subject to be treated, and the like. The application may also allow a third-party responder to connect live to a remote medical professional who can guide the user on the next steps. The application may be used with artificial intelligence to perform triage by itself, e.g., in the case of a mass casualty or injury scenario.

The application can include a geolocation feature that displays the global position (e.g., using GPS tracking) of the user on a map. The map may also show the position of other users (e.g., team members) using a peripheral device, application, and/or bracelet. The application may have a screen that displays user information, such as name, roster, unit, allergies, an identification number (e.g., a social security number), and blood type. When an ID card is scanned at the beginning of a mission, the application can automatically load the various user information in order to personalize the bracelet and/or peripheral device for a specific user. The application may have a screen that displays system status, such as power, connectivity signal, and status of the impact and vital signs monitoring (VSM) sensors.

The application may have a screen that displays the system settings, which can be adjusted by the user. The sensors and alerts can be turned on or off and the level of sensitivity can be adjusted on a discrete or sliding scale (e.g., from 1 to 10, or from sensitive to robust). The application may include a screen that includes a map that displays the position of each member of a team, e.g., as an encircled dot. In the event that a member has been injured, the dot may change to a different indicator, e.g., a color change, such as red, to alert the other team members. The components of the system interact with the peripheral device to signal (e.g., via the application or radio) to alert the other team members (FIG. 8). One of the other team members may then request a third party responder (e.g., medical evacuation team) if the injury is serious. Exemplary applications are described, e.g., in US Pub. No. US 20200237318, the disclosure of which is hereby incorporated by reference in its entirety.

Once the application running on or accessible from the peripheral device identifies or senses that the user wearing the bracelet has been injured, the application can activate multiple features to transmit information specific to the injured user to appropriate personnel. For example, the application may include a screen that shows a map displaying the position of an injured user. Each team member can click an icon on the map to open a user information card (e.g., tactical combat casualty care (TCCC) card) corresponding to the injured team member or a K9 TCCC for an injured dog. Information, such as the location (e.g., arm, torso, and chest) and force (e.g., 10 pN-1000 pN) of the impact, VSM information (e.g., heart rate (beat/min), respiration rate (respirations/min)), and the time or point of impact (TOI/POI) time stamp may be displayed. This information may also be input into the user information card (e.g., TCCC) via an audio or visual recording device that electronically fills out the TCCC based on the input message. The application may also include a medical evacuation request icon to initiate a medical evacuation request, e.g., using medical evacuation request form. The application may have a screen to input information. This information may be tabulated in an electronic user information card (e.g., electronic TCCC card) for easy visual consumption by a third party responder. For example, the application may include a screen with various information boxes that are pre-populated (e.g., evacuation category, name, date, unit, battle roster, identification number (e.g., social security number), time, and allergies), but can be overwritten (e.g., by the injured person or a third party responder), if necessary or desired. Additionally, the screen may display a continuous live transmission view of the injured team member's vital signs, such as heart rate and respiration rate.

The application may include a mode to display and/or allow input of a cause of the injury (e.g., artillery, burn, fall, grenade, gunshot wound (GSW), improvised explosive device (IED), landmine, motor vehicle collision (MVC), rocket propelled grenade (RPG), and other). The application may include a mode to display and/or allow input of the location and type of injury on the user. The application may include a mode to display and/or allow input by a user (either the injured user, a team member, or a third party) where on the body (e.g., right, left, arm, or leg) the injury occurred. The application may include a mode to display and/or allow input of signs and symptoms of the user, including time, blood pressure, pulse and oxygen saturation, alert, voice, pain, unresponsive (AVPU), and pain scale (e.g., 1-10) following an injury. The application may include a mode to display and/or allow input of a treatment performed (e.g., by a third party responder) on the injured user, such as the use of an extremity tourniquet, junctional tourniquet, pressure dressing, hemostatic dressing, intact device, cricothyrotomy (CRIC), supraglottic airway (SGA) device, nasopharyngeal airway (NPA) device, endotracheal tube, oxygen, chest tube, chest seal, or needle. The application may include a mode to display and/or allow input of a blood treatment performed on the injured user, such as fluid and blood product, and name, volume, route, and time. The application may also include a mode to display and/or allow input of medicines administered to the injured user, such as analgesics, antibiotics, or other therapeutics, and name, dose, route, and time, and/or treatments administered, such as combat pill pack, eye shield (e.g., right or left), splint, or hypothermia prevention. The application may further include a mode to display and/or allow input of additional notes.

Once the information is filled out using the application, the application provides further functionality allowing the injured user or a third party responder to request medical evacuation and/or to send the user information card (e.g., an electronic TCCC card) to another responder or medical evacuation team. If the user or a responder determines that a medical evacuation is required, the user can input location (e.g., GPS location) by selecting, e.g., XYZ grid coordinates on a map. The user requesting medical evacuation can also input a specific radio frequency and call sign and suffix that he is using and indicate number of injured users or others, e.g., patients (PXT) by precedence, (e.g., urgent, urgent-surgery required, priority, routine, and convenience). Furthermore, the application includes programming to allow the user to request special equipment, such as a hoist, extraction equipment, or a ventilator. The application may include an entry to indicate the number of injured users or others, e.g., patients (PXT) by type (e.g., ambulatory). The application may also include a feature to indicate the wartime security of the user zone (e.g., no enemy troops, possible enemy, enemy in area/proceed with caution, and enemy in area/armed escort required). The application may also include a feature to indicate method of marking (e.g., panels, pyrotechnic signal, smoke signal, or no signal). The user requesting medical evacuation can indicate nationality and status (e.g., US military, US civilian, non-US military, non-US civilian, and enemy prisoner of war (EPW)) of an injured user or other personnel. Additionally, the user requesting medical evacuation may indicate the wartime nuclear, biological, or chemical (NBC) contamination status (e.g., chemical, biological, radiological, and nuclear). If using, e.g., an ATAK platform and a TCCC card, the application can process all of the sensor data and the information inputted and/or gathered via the GUI onto an electronic TCCC card to summarize all of the information for a third party responder. If not using an ATAK platform, the application can process all of the sensor data and the information inputted and/or gathered via the GUI onto a user information card (e.g., electronic user information card). The application may also output the data onto a medical evacuation request form to summarize all of the information for a third party responder.

### Graphical User Interface (GUI)

The peripheral devices described herein may include a GUI that displays, e.g., various sensor information and/or health indicia associated with a user wearing the bracelet. The sensor information and/or health indicia may be collected by the sensors (e.g., physiologic or biometric sensors), e.g., on the bracelet, and processed by the application. The application outputs the information to the GUI. The application can be configured to output information regarding the status of the user or bracelet, such as stored energy level or remaining battery power or on/off status. The application can also output data to the GUI regarding information about the features or stimuli detected by the sensors of the bracelet. The GUI may be an LED device or other monitor, tablet, or smartphone, or the like, as long as it is capable of displaying or depicting information to a user. The GUI may include holographic or projected display (e.g., smart glasses, such as GOOGLE® glass). The GUI may be connected (e.g., wired, or wirelessly) to or integrated with the bracelet or to the peripheral device. The GUI may be the peripheral device or part of the peripheral device.

### Information Processing Unit

The peripheral device and/or the bracelet may include an information processing unit. The information processing unit may include one or more of a processor, controller (e.g., a microcontroller), a programmable memory, and/or a data storage system (e.g., a flash memory system) which can be used to record data from sensor inputs. The unit processes the signals received from the geolocation and physiological sensors, such as vital signs monitoring (VSM) sensors, temperature sensors, moisture sensors, and blood pressure sensors. Depending on the outcome of the computation in interaction with the program stored on the memory, the unit may then alert a third party responder (e.g., medical responder or team member). The information processing unit may also trigger the transmission of data (such as a distress signal) via a data transmission unit. The information processing unit may be incorporated into the peripheral device and programmed to interact with the application or vice versa. The information processing unit may be a smartphone (e.g., ANDROID™). Alternatively, the information processing unit may be part of a cloud-based or internet-based system (e.g., a remote server). The information processing unit may be disposed in a module (e.g., an electronics module) on top of the bracelet (FIG. 2).

The information processing unit may be configured to identify the nature of an injury or health status by analyzing sensor data. For example, by sensing the heart rate and blood pressure of a subject, the information processing unit may be able to identify if a subject is in need of immediate treatment. By coupling this data with the specific location of the user, indicia is provided that can alert the user and/or a third party responder as to the identity, nature, and severity of the issue.

The information processing unit may be configured to integrate data obtained from multiple different types of sensors to provide essential physiological information about the health status of a user. By integrating various sensor data, the information processing unit provides increased situational awareness for the user and/or a third party responder. For example, if the orientation and acceleration sensors determine that the user is still moving, the third party responder receiving this sensor data information may determine that the person is not in need of immediate attention. However, if the orientation and acceleration sensors determine that the user is not moving and/or is in a prone position, a third party responder receiving this sensor data information may determine that the user may be in need of immediate attention. The sensors can use information such as injury location, time passed since impact, and biometric data (e.g., breathing rate) to signal for remote triage, e.g., to categorize response in order of importance.

The information processing unit may process the data to assign a health state to the subject wearing the bracelet, e.g., based on the physiological data. The health states may be determined using various physiological data, such as movement, respiration rate, blood pressure, heart rate, perfusion, and mental cognition. The method may include assigning one of four health states to the subject. The four health states may include, for example, (i) black or deceased or near deceased (e.g., no respiration and/or pulse detected); (ii) red or immediate assistance required (e.g., respiration detected (e.g., >30 breaths per minute), unconscious, perfusion detected (e.g., >2 capillary refill test (CRT)), no radial pulse, uncontrolled bleeding, decreased mental cognition, such as unable to follow simple commands); (iii) yellow/orange or delayed response required (e.g., injured or uninjured, no immediate care required or low risk of significant deterioration with delayed treatment); and (iv) green or minor or walking wounded (e.g., ambulatory but injuries are minor). The method may include triaging treatment of the subject by a third-party responder based on the health state of the subject. The health states may be denoted by a colorimetric LED or a plurality of colorimetric LEDs on the bracelet, where each LED has a different color (FIG. 6). The information processing unit may alert a third-party responder to treat the subject. The information processing unit may use artificial intelligence, an algorithm, or software application to assign a health state based on compiling the physiological data from the sensors. Furthermore, the information processing unit can track any changes in the physiological data and change the health state accordingly if a corresponding change is detected (e.g., the subject's heart rate, respiration rate, or blood pressure drops). Alternatively, a third party responder could manually change the health state, e.g., by inputting this information on the peripheral device. The triaging may be performed according to the Simple triage and rapid treatment (START) protocol (see, e.g., remm.hhs.gov/startadult.htm).

In some instances, by combining the sensor data, the information processing unit can determine false positives and false negatives by corroborating the severity of the injury between multiple types of sensors. For example, if a heart rate sensor does not detect a heart rate of the user, but the geolocation or GPS sensor detects movement of the user and/or an upright, standing position of the user, the device can notify the user and/or a third party responder that the absence of a heart rate signal may be false or in error.

### Operation

The following section describes the function of a bracelet for use with a human subject (see, e.g., FIGS. 1-5). The bracelet includes a geolocation sensor and one or more physiological sensors. The physiological sensors may be selected from a heart rate sensor, an oxygen saturation sensor, a blood pressure sensor, a respiratory rate sensor, a temperature sensor, a vital sign monitoring (VSM) sensor, an impact detection sensor, a position sensor, a contact sensor, a hydration sensor, an electrolyte sensor, and a movement sensor. The geolocation sensor and/or the one or more physiological sensors may be integrated within the bracelet, e.g., as opposed to being housed in a separate module. The geolocation sensor and/or the one or more physiological sensors may be internal sensors. The geolocation sensor and/or the one or more physiological sensors may be external sensors, e.g., embedded on an external portion of the bracelet. The device may also be connected, e.g., wired or wirelessly, to other external sensors (e.g., off the shelf physiological sensors), such as vital sign monitoring sensors, or those present in a wearable device as described herein. The geolocation sensor and/or the one or more physiological sensors may be configured to be activated upon contact with a user and/or deformation of the bracelet.

The bracelet may be adorned on a subject, e.g., by slapping the bracelet onto a wrist of the subject, and the bracelet coils onto the wrist (FIG. 7). Upon contact with the subject, the various sensors may be activated. The bracelet may further include an orientation sensor. The orientation sensor may provide an audio or visual stimulus when the bracelet is adorned in a correct orientation on a subject. The visual stimulus may include, for example, a colorimetric light emitting diode (LED). For example, the colorimetric LED may turn from red to orange or green to indicate that the bracelet is adorned in the correct orientation on the subject. The correct orientation may be an orientation in which the PCB or main electronics module is positioned on top of the wrist, like a watch. In another embodiment, the bracelet may be correctly oriented when a heart rate sensor is positioned near the radial artery on the wrist to appropriately track the subject's pulse.

The sensors may collect various indicia from the subject, such as heart rate, heart rate variability, oxygen saturation, blood pressure, respiratory rate, temperature, vital signs, injury status, position, body orientation, hydration status, and movement status. The bracelet is wirelessly connected to an end-user peripheral device. The end-user peripheral device may include a display; one or more processors coupled to the display; and a non-transient memory storing instructions that, when executed by the one or more processors, causes the one or more processors to perform one or more operations. The operations may include rendering a graphical user interface in the display; processing sensor data to produce physiological data; receiving an input of physiological data to the graphical user interface; and/or displaying the physiological data on the graphical user interface.

The peripheral device can then present the physiological data regarding the health state of the subject wearing the bracelet. The end-user peripheral device may include a graphical user interface that displays the physiological data, such as one or more of heart rate, heart rate variability, oxygen saturation, blood pressure, respiratory rate, temperature, vital signs, injury status, position, body orientation, and hydration status, movement status. The graphical user interface may display a geographical location of the subject, e.g., on a map.

The peripheral device includes an information processing unit that analyzes the various physiological indicia from the health sensors. Based on this information, the information processing unit (or the third party responder) can assign a health state to the subject wearing the bracelet and present this information on the graphical user interface. The four health states may include, for example, (i) black or deceased or near deceased (e.g., no respiration and/or pulse detected); (ii) red or immediate assistance required (e.g., respiration detected (e.g., >30 breaths per minute), unconscious, perfusion detected (e.g., >2 capillary refill test (CRT)), no radial pulse, uncontrolled bleeding, decreased mental cognition, such as unable to follow simple commands); (iii) yellow/orange or delayed response required (e.g., injured or uninjured, no immediate care required or low risk of significant deterioration with delayed treatment); and (iv) green or minor or walking wounded (e.g., ambulatory but injuries are minor). Based on the health state, the peripheral device alerts the third-party responder as to the severity of injury. The third party responder can then triage treatment of the subject by that third party responder or another third-party responder based on the health state of the subject. In the event of a catastrophic injury, the device may trigger an emergency alert to signal for an immediate rescue.

### Operation With a Non-Human Animal

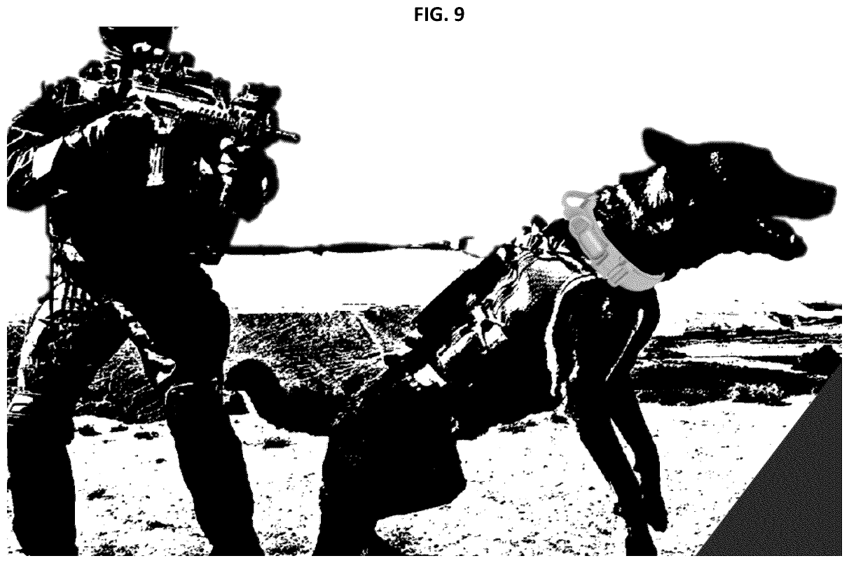

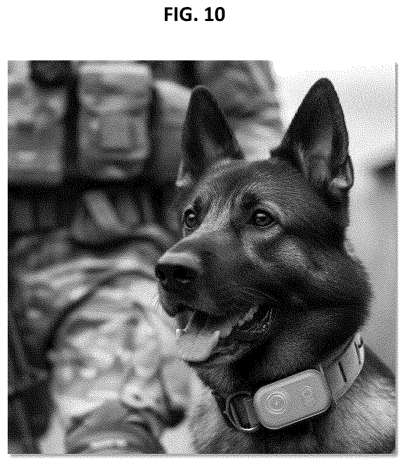

The following section describes the function of a bracelet configured as a collar for use with a non-human subject, such as a dog (see, e.g., FIGS. 9-12). The collar includes a geolocation sensor and one or more physiological sensors. The physiological sensors may be selected from a heart rate sensor, an oxygen saturation sensor, a blood pressure sensor, a respiratory rate sensor, a temperature sensor, a vital sign monitoring (VSM) sensor, an impact detection sensor, a position sensor, a contact sensor, a hydration sensor, an electrolyte sensor, and a movement sensor. The geolocation sensor and/or the one or more physiological sensors may be integrated within the collar, e.g., as opposed to being housed in a separate module. The geolocation sensor and/or the one or more physiological sensors may be internal sensors. The geolocation sensor and/or the one or more physiological sensors may be external sensors, e.g., embedded on an external portion of the collar. The geolocation sensor and/or the one or more physiological sensors may be configured to be activated upon contact with a user and/or deformation of the collar. The collar may also be connected, e.g., wired or wirelessly, to other external sensors (e.g., off the shelf physiological sensors), such as vital sign monitoring sensors or those present in a wearable device as described herein.

The collar may be adorned on a dog, e.g., by fastening the collar using a buckle or clasp (FIG. 9). The various sensors may be activated, e.g., upon contact with the dog or by external activation. Alternatively, the sensors may be ready to collect physiological data without activation (e.g., the sensors are powered on). The collar may further include an orientation sensor. The orientation sensor may provide an audio or visual stimulus when the collar is adorned in a correct orientation on a subject (e.g., dog). The visual stimulus may include, for example, a colorimetric light emitting diode (LED). For example, the colorimetric LED may turn from red to orange or green to indicate that the collar is adorned in the correct orientation on the subject (e.g., dog). The correct orientation may be an orientation in which the PCB or main electronics module is positioned on top of the wrist, like a watch. In another embodiment, the collar may be correctly oriented when a heart rate sensor is positioned near an artery on the neck to appropriately track the subject's pulse.

The collar may be configured to provide an audio, visual, mechanical, or electronic stimulus to the animal (e.g., to control the animal's behavior from the handler or provide a command, e.g., to recall the animal). The collar may contain an output device, such as a speaker or an alarm. The output device (e.g., speaker) may be used to communicate with the animal or handler, e.g. to send an alarm. For example, a text, video, or audio message can be input from a peripheral device and sent to the animal wearing the collar or to a third party responder, such as a message providing treatment instructions.

The sensors may collect various indicia from the subject (e.g., dog), such as heart rate, heart rate variability, oxygen saturation, blood pressure, respiratory rate, temperature, vital signs, injury status, position, body orientation, hydration status, and movement status. The collar is wirelessly connected to an end-user peripheral device. The end-user peripheral device may include a display; one or more processors coupled to the display; and a non-transient memory storing instructions that, when executed by the one or more processors, causes the one or more processors to perform one or more operations. The operations may include rendering a graphical user interface in the display; processing sensor data to produce physiological data; receiving an input of physiological data to the graphical user interface; and/or displaying the physiological data on the graphical user interface.

The peripheral device can then present the physiological data regarding the health state of the subject (e.g., dog) wearing the collar. The end-user peripheral device may include a graphical user interface that displays the physiological data, such as one or more of heart rate, heart rate variability, oxygen saturation, blood pressure, respiratory rate, temperature, vital signs, injury status, position, body orientation, hydration status, and movement status. The graphical user interface may display a geographical location of the subject (e.g., dog), e.g., on a map.

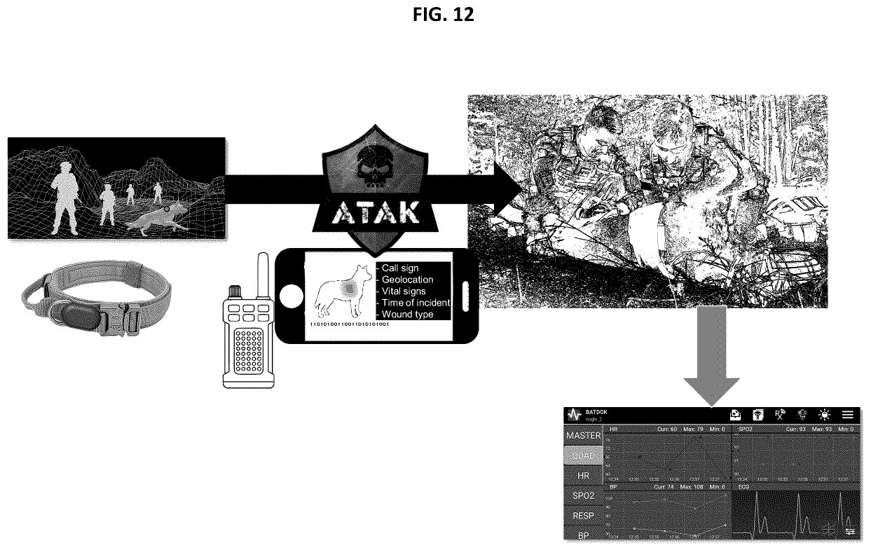

The peripheral device includes an information processing unit that analyzes the various physiological indicia from the health sensors. Based on this information, the information processing unit (or the third party responder) can assign a health state to the subject wearing the collar and present this information on the graphical user interface, such as with color coding. The four health states may include, for example, (i) the color black for deceased or near deceased (e.g., no respiration and/or pulse detected); (ii) the color red for immediate assistance required (e.g., respiration detected (e.g., >30 breaths per minute), unconscious, perfusion detected (e.g., >2 capillary refill test (CRT)), no radial pulse, uncontrolled bleeding, decreased mental cognition, such as unable to follow simple commands); (iii) the color yellow/orange for delayed response required (e.g., injured or uninjured, no immediate care required or low risk of significant deterioration with delayed treatment); and (iv) the color green for minor or walking wounded (e.g., ambulatory but injuries are minor). Based on the health state, the peripheral device alerts the third-party responder as to the severity of injury. The third party responder can then triage treatment of the subject by that third party responder or another third-party responder based on the health state of the subject (FIG. 12). In the event of a catastrophic injury, the device may trigger an emergency alert to signal for an immediate rescue.

### EXAMPLES

### Example 1. Responding to an Injured Team Member

Each member of a team of four operators (users 1-4) is in a battlefield mission. Upon attack of their troop, one of the members alerts their team leader that they are under attack. The team leader pulls out a bracelet and slaps it onto the wrist of each of the four members. Each bracelet has a unique identifier to identify the wearer and activates upon contact with the user.

The bracelet begins collecting various physical indicia and the precise geographical location of the subject. Each bracelet is configured to interact with (e.g., via a wired connection or Bluetooth or other wireless connection) a peripheral device operated by a senior commander at a remote location. The peripheral device is configured to run a smartphone application that processes sensor data obtained from sensors on the bracelet of each of the four team members.

One of the team members is wounded by an impact. The heart rate and blood pressure begin to drop precipitously, and these indicia alert the senior commander on his peripheral device that the team member is severely injured. Following the injury, the system can send out the user information and location to a third party responder and can begin to continuously transmit vital sign information, including heart rate, heart rate variability, blood pressure, and respiration rate.

A medic receives notice of the four team members but recognizes that one is in serious need of treatment. When the medic arrives at the injured team member, he can click on the location of the user on the map shown on his peripheral device to obtain information about the injured user. The medic can begin to input details about the injured user on the touch screen of his user interface. The medic can identify the type of injury (e.g., one caused by an RPG) and can input this information into the application running on his peripheral device. The application can begin to fill out an electronic user information card with the information input by the responder. The medic may identify a second injury on user 1 (e.g., an injury to the right arm) and can perform some routine medical tests to check the injured user's blood pressure and pain scale. The medic can apply a pressure dressing to the arm wound and can administer an antibiotic to the injured user to prevent rapid onset of infection. After stopping the bleeding in the torso and the right arm, the medic can put the arm in a splint and can enter an additional note that the injured team member is diabetic.

The additional sensors (e.g., biometric sensors) of the bracelet can also be used to sense if the condition of the injured user deteriorates. The blood pressure can be continuously monitored and displayed on the GUI and of the peripheral device. An alert can sound when the blood pressure drops to a dangerous level. The medic can immediately recognize that a medical evacuation is necessary. The medic can input the GPS location of the injured user in the application by clicking on the map on the user interface and can transmit the electronic user information card of the injured team member to another user or a third-party responder, such as a medical evacuation team. The medic can alert the medical evacuation team that the situation is urgent, a ventilator is required for the injured team member, and that an enemy troop is located nearby, requiring the evacuation team to proceed with caution. The medic can mark the pickup zone with a panel and can send a finalized alert message. The medic can input his radio frequency for an additional line of communication while awaiting evacuation. The medical evacuation team may then arrive in a helicopter prepared with the necessary treatment accessories based on the user's injuries. The evacuation team can also be equipped with insulin to treat the injured member's diabetes. The medical team can resuscitate the user, if necessary, and can transport him to the local base hospital.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

The complete disclosures of all patents, patent applications including provisional patent applications, publications including patent publications, and nonpatent publications cited herein are incorporated by reference.

Other embodiments are within the claims.

1. A bracelet comprising a geolocation sensor and one or more physiological sensors selected from a heart rate sensor, an oxygen saturation sensor, a blood pressure sensor, a respiratory rate sensor, a temperature sensor, a vital sign monitoring (VSM) sensor, an impact detection sensor, a position sensor, a contact sensor, a hydration sensor, an electrolyte sensor, and a movement sensor, wherein the geolocation sensor and/or the one or more physiological sensors is integrated within the bracelet.

2. A bracelet comprising a geolocation sensor and one or more physiological sensors selected from a heart rate sensor, an oxygen saturation sensor, a blood pressure sensor, a respiratory rate sensor, a temperature sensor, a vital sign monitoring (VSM) sensor, an impact detection sensor, a position sensor, a contact sensor, a hydration sensor, an electrolyte sensor, and a movement sensor, wherein the geolocation sensor and/or the one or more physiological sensors is configured to be activated upon contact with a user and/or by deformation of the bracelet.

3. The bracelet of claim 1, wherein the bracelet comprises an extended configuration and a coiled configuration.

4. The bracelet of claim 3, wherein the bracelet comprises:
(a) a first end and a second end opposite the first end, wherein the first end of the bracelet is configured to partially overlap with the second end of the bracelet upon coiling around an appendage of a user; or
(b) a layer comprising flexible material shaped with a width-wise arc, wherein the layer is disposed in a linear orientation in the extended configuration and in a coiled orientation in the coiled configuration, and wherein the bracelet is configured to adopt the coiled orientation by deforming the width-wise arc.

5-7. (canceled)

8. The bracelet of claim 4, further comprising an outer layer that covers the layer comprising the flexible material.

9-11. (canceled)

12. The bracelet of claim 1, wherein the bracelet is configured to be worn on a neck, an arm, or a leg of a subject.

13. (canceled)

14. The bracelet of claim 1, wherein the geolocation sensor comprises a global positioning satellite module.

15. The bracelet of claim 1, further comprising one or more of a button that activates the geolocation sensor and/or the one or more physiological sensors, a transmitter that transmits data from the geolocation sensor and/or the one or more physiological sensors, a radio node, an orientation sensor, and an audio or visual recording device.

16-17. (canceled)

18. The bracelet of claim 15, wherein:
(a) the transmitter is a radio frequency transmitter, a cellular transmitter, a WiFi transmitter, an adaptive network topology (ANT) transmitter, a satellite transmitter, or a Bluetooth transmitter;
(b) the radio node is configured to be integrated within a local area network (LAN) topology; and/or
(c) the orientation sensor is configured to provide an audio, visual, or mechanical stimulus when the bracelet is adorned in a correct orientation on a subject.

19. The bracelet of claim 15, wherein the data is compiled into a digital casualty care profile card.

20-21. (canceled)

22. The bracelet of claim 18, wherein the LAN topology is a mesh network.

23-27. (canceled)

28. The bracelet of claim 1, wherein the bracelet is configured to interact with an end-user peripheral device and/or another bracelet.

29-32. (canceled)

33. A system comprising the bracelet of claim 1 and an end user peripheral device, wherein the bracelet is wired or wirelessly connected to the end-user peripheral device.

34. The system of claim 33, wherein the end-user peripheral device comprises:
a display;
one or more processors coupled to the display; and
a non-transient memory storing instructions that, when executed by the one or more processors, causes the one or more processors to perform operations including:
rendering a graphical user interface in the display;
processing sensor data to produce physiological data;
receiving an input of physiological data to the graphical user interface; and/or
displaying the physiological data on the graphical user interface;
wherein the peripheral device is configured for wired or wireless communication with one or more sensors of the bracelet.

35. The system of claim 34, wherein the physiological data are selected from one or more of heart rate, heart rate variability, oxygen saturation, blood pressure, respiratory rate, temperature, vital signs, injury status, position, body orientation, and movement status.

36. (canceled)

37. The system of claim 33, comprising a plurality of the bracelets, each of which is independently wirelessly connected to the end-user peripheral device.

38. A method for presenting physiological and/or geolocation data regarding the status or health state of a subject wearing the bracelet of claim 1, the method comprising acquiring the physiological data from the geolocation sensor and the one or more physiological sensors of the bracelet and presenting the physiological data on an end-user peripheral device.

39-41. (canceled)

42. The method of any one of claims claim 38, further comprising assigning a health state to the subject wearing the bracelet based on the physiological data, triaging treatment of the subject by a third-party responder, and/or alerting a third-party responder to treat the subject.

43-47. (canceled)

48. The method of claim 38, wherein the bracelet comprises a mechanical stimulus and/or an audio or visual recording and/or transmitting device and the method comprises presenting an audio, visual, or mechanical message on the peripheral device or the bracelet.

49. A kit comprising the bracelet of claim 1.

50-52. (canceled)
